(19) United States
(12) Patent Application Publication
(10) Pub. No.: US 2010/0076315 A1
(43) Pub. Date: Mar. 25, 2010

(54) METHOD AND APPARATUS FOR HANDS-FREE ULTRASOUND

(75) Inventors: Ramon Q. Erkamp, Purdys, NY (US); Eric V. Cohen-Solal, Ossining, NY (US); Balasundara I. Raju, Tarrytown, NY (US); Jose M. I. Azevedo, Mahopac, NY (US)

Correspondence Address: PHILIPS INTELLECTUAL PROPERTY & STANDARDS, P. O. Box 3001, BRIARCLIFF MANOR, NY, 10510, US

(73) Assignee: KONINKLIJKE PHILIPS ELECTRONICS N. V., Eindhoven (NL)

(21) Appl. No.: 12/442,710

(22) PCT Filed: Sep. 10, 2007
(86) PCT No.: PCT/US07/77979

§ 371 Date: Mar. 25, 2009

Related U.S. Application Data
(60) Provisional application No. 60/827,476, filed on Sep. 29, 2006.

(51) Int. Cl. A61B 8/14

(52) U.S. Cl. 600/459

(57) ABSTRACT

A skin-mounted device (10) comprises an acoustic module (24) and an attachment assembly (16). An acoustically coupling layer (34) affixes and acoustically couples the module (24) to a skin (18).

The present application relates to diagnostic arts. It finds particular application in relation to ultrasound monitoring and will be described with particular reference thereto. However, it is to be appreciated that the following will also find application in conjunction with ultrasound imaging, testing, treatment, and the like.

Ultrasound systems are valuable diagnostic tools for providing in real time critical information about the patient's condition such as flow of blood, heart beat, tissue movements, and the like. Typically, the ultrasound diagnostic systems use non-invasive technology based, for example, on the Doppler effect. In such systems, high accuracy of measurements is combined with simplicity of diagnostic procedures. Typically, a medical professional performs ultrasound by using a manual probe or transducer by applying a pressure and changing the position of the probe.

Additionally, the Doppler systems can be used to detect the patient's pulse and measure blood flow in emergency situations, during surgery, in ICU, and the like. In such situations, a presence of a medical professional who is dedicated to the ultrasound machine is required in addition to other medical personnel. Similarly, in many therapeutic applications of ultrasound, an operator is needed to hold the transducer at a specific part of the body. This is costly and, as studies have shown, causes undesirable health deteriorations in the medical personnel such as repetitive stress injury due to the need to apply the constant pressure on the transducer. A direct attachment of the transducer to the patient's skin could avoid the extra workload on medical personnel and make ultrasonic monitoring more attractive.

One approach is to attach an ultrasound transducer to the patient's skin with an adhesive bandage or the like. However, such technique does not soundly acoustically couple the ultrasound transducers with the patient's skin. Another approach is to place a gel pad between the transducers and the skin. Yet another approach is to spread a conventional acoustic coupling gel to the skin and apply the ultrasound transducers over it. The acoustic pad is disadvantageous because air pockets are defined between the pad and fissures in the skin which cause excessive acoustic reflection. Acoustic coupling gel is disadvantageous because it is slippery and might permit movement of the transducer relative to the skin.

The present application provides new and improved methods and apparatuses which overcome the above-referenced problems and others.

In accordance with one aspect, a skin-mounted device is disclosed which comprises an acoustic module and an attachment assembly. An acoustically coupling layer affixes and acoustically couples the module to the skin.

In accordance with another aspect, a method of acoustic monitoring is disclosed. An acoustic module is affixed and acoustically coupled with a patient's skin with an acoustic coupling layer. Acoustic signals are transmitted and received with the module to monitor a physiological parameter of the patient.

One advantage resides in a hands-free mechanism which affixes an ultrasound sensor to the skin.

Still further advantages of the present invention will be appreciated to those of ordinary skill in the art upon reading and understanding the following detailed description.

The invention may take form in various components and arrangements of components, and in various steps and arrangements of steps. The drawings are only for purposes of illustrating the preferred embodiments and are not to be construed as limiting the invention.

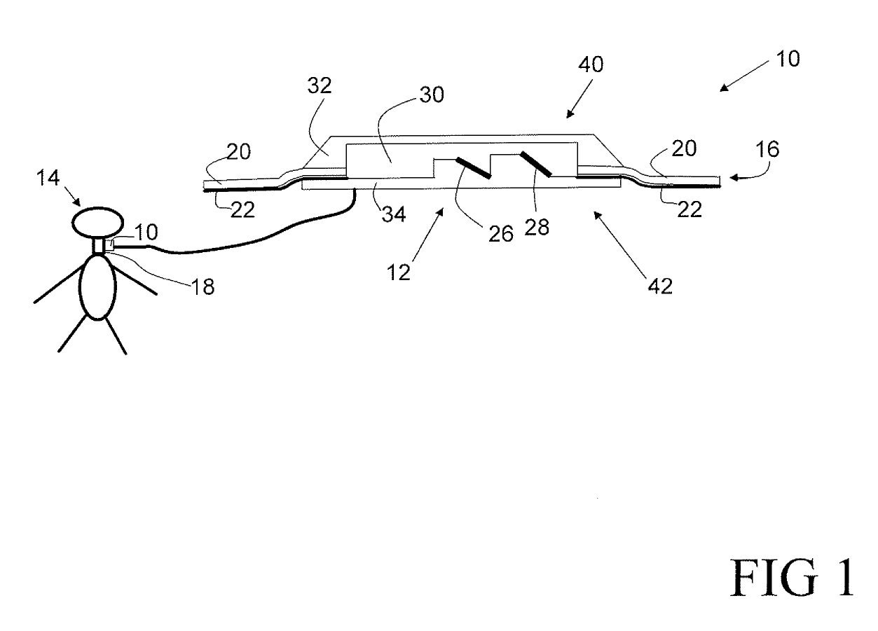

FIG. 1 is a diagrammatic illustration of an ultrasound system;

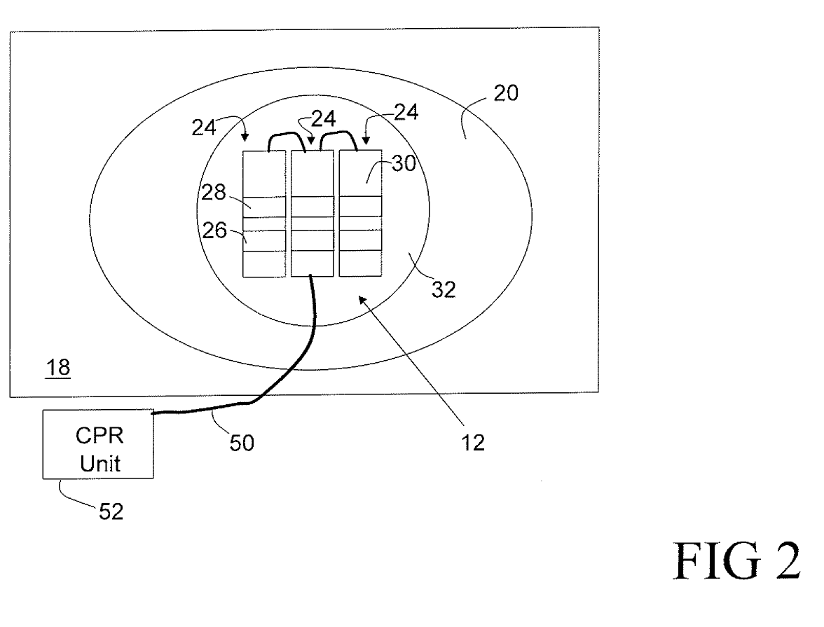

FIG. 2 is a diagrammatic illustration of a bottom view of an ultrasound assembly;

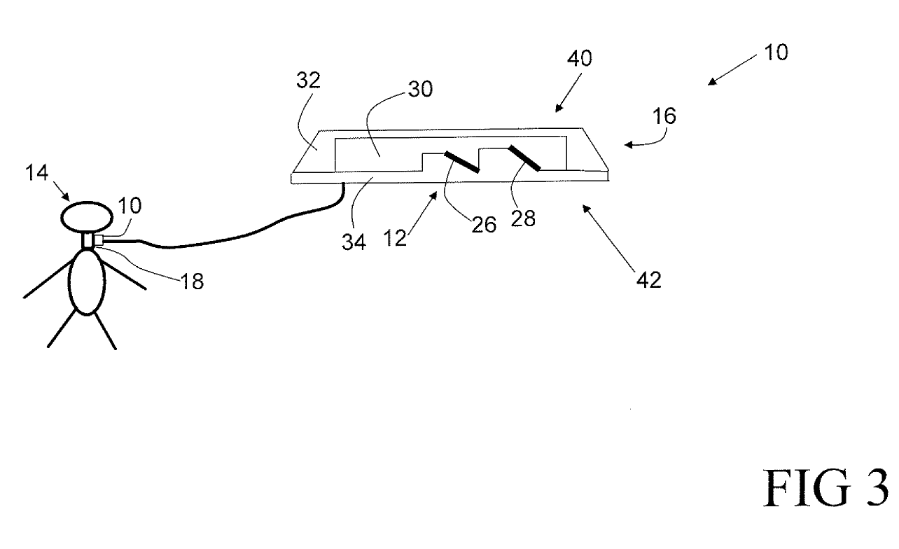

FIG. 3 is a diagrammatic illustration of another embodiment of an ultrasound system; and

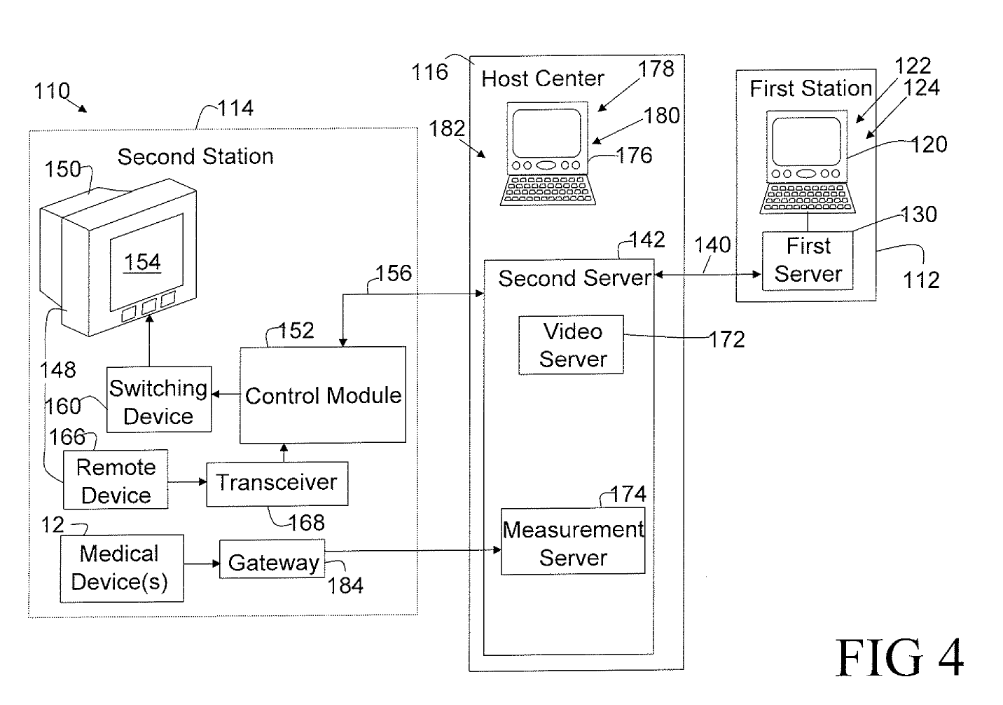

FIG. 4 is a diagrammatic illustration of a monitoring system.

With reference to FIGS. 1 and 2, an ultrasound assembly 10 includes a medical, monitoring or biometric device 12 such as a Doppler effect based ultrasound sensor which, for example, detects, measures, and/or monitors a physiological parameter. The examples of the physiological parameter are flow of blood, heart beat, and tissue movements. The medical device 12 is attached to a patient 14 via an attachment assembly 16 which securely attaches the medical device 12 to a base surface or skin 18 of the patient 14. More specifically to the first embodiment, the attachment assembly 16 includes a pad 20, such as a pediatric foam pad, with a layer 22 of adhesive material that adheres the pad 20 with the ultrasound sensor 12 to the skin of the patient. In one embodiment, the ultrasound sensor 12 includes a piezoelectric transducer including an array of piezoelectric modules, elements or crystals 24. Each piezoelectric module 24 includes a transmitter 26 and a receiver 28 mounted to a former or support 30. The former 30 holds each module element 24 sufficiently rigid so that the orientation between each pair of transmitter and receiver of each element 24 is fixed. Of course, it is contemplated that the module includes a transceiver. The piezoelectric modules 24 are interconnected with a stiff, but sufficiently flexible covering layer 32 so that the transducers can be contoured to the contour of the patient. Of course, for imaging transducer arrays, all of the transducers need to be kept in a preselected, known relationship to each other. Although a piezoelectric transducer is illustrated in the exemplary embodiment, an ordinary person skilled in the art will appreciate that any other type of component or material that exhibits piezoelectric attributes may be used so that when the material is energized it produces a mechanical effect. Any other ultrasound transducers such as for example CMUT (capacitive micromachined ultrasonic transducer) may be used as well.

The modules 24 are enclosed in the flexible covering layer 32, which in one embodiment, is a flexible silicone material. For example, the covering layer 32 includes a layer of a rigid adhesive material such a T2® silicone that covers a top or first surface 40 of the ultrasound assembly 10. The attachment assembly 16 also includes an acoustic coupling layer 34 which is disposed at a lower or second surface 42 of the ultrasound assembly 10 to make direct contact with the skin 18. For example, the coupling layer 34 includes a layer of an elastomeric material which is sufficiently tacky or sticky so that it adheres to the skin 18 and, which, although sufficiently firm so as to prevent transducer slippage, is sufficiently fluid so that it penetrates and fills fissures in the skin, eliminating air pockets. The material of the coupling layer 34 has an acoustic transmissivity which transmits the ultrasound signals at substantially the speed of sound in human tissue (water), with reasonably low attenuation and an acoustic impedance near that of tissue. An example of a suitable material is Sylgard® which is a dielectric gel manufactured by Dow Corning. Such gel represents a special encapsulant that can cure to an extremely soft material such as skin. The cured gel retains much of the stress, relief and self healing qualities of the liquid while providing the dimensional stability of an elastomer. The gel is formulated and cured such that the surface of the cured gel is naturally tacky which allows the gel to gain physical adhesion without the need for primers.

In one embodiment, the coupling layer 34 is substantially rigid with the strong adhesive qualities. In another embodiment, the sensor is strongly pushed onto the skin with a peripheral adhesive attachment.

With continuing reference to FIG. 2, conductor 50 connects the ultrasound sensor 12 to a monitor unit, such as a CPR unit 52. Optionally, the ultrasound assembly 10 can include a battery, an ultrasound, a transmit/receive controller, and a wireless transmitter for wireless communications such as body coupled communications, Bluetooth, or the like. The circuitry for interpreting the ultrasound echoes, such as the circuitry for connecting the echo data into pulse rate, can be mounted in part or in full on the ultrasound assembly 10. As another option, a wireless communication and battery module can be tethered to the ultrasound assembly.

Directly adhering the ultrasound assembly 10 onto the patient 14 prevents movement of the ultrasound sensor 12 while sensing the Doppler energy in, for example, the carotid artery. The ultrasound assembly 10 is isolated from sensing false Doppler energy generated because of transducer movement. Further, the adhesiveness of the attachment assembly 16 provides the medical personnel with a degree of freedom. Specifically, the medical professional may place the ultrasound assembly 10 on the patient 14 without having to hold it in place while taking the pulse. Future pulse checks can be performed without further personal attention to the transducer assembly. The large adhesive contact area reduces motion due to tension in the sensor cable. The Sylgard® coupling material provides good acoustic contact while eliminating air bubbles at the skin interface, and is also non-perishable so that long term storage is not an issue. The lower sound speed in this material causes beam refraction allowing for a flatter patch design because the crystals do not have to be angled as steep.

The refraction increases the ultrasound beam angle out of the sensor which is needed to get a strong Doppler component. This makes the overall design even more like a flat patch because the piezo elements do not have to be steeply angled to get a steep exit angle of the beam from the sensor.

With reference to FIG. 3, the piezoelectric modules 24 are disposed between the covering and acoustic coupling layers 32, 34. The pad 20 with the adhesive layer 22 of FIGS. 1 and 2 is omitted. The acoustic coupling layer 34 includes a sticky elastomeric silicone material such as Sylgard®. The flexible covering layer 32 includes a rigid non-sticky silicone material such as T2®. In another embodiment, the covering and acoustic coupling layers 32, 34 both include elastomeric silicone material such as Sylgard®, The covering layer 32 includes a thin outer layer of non sticky material such as a Teflon layer to facilitate manual handling.

The Sylgard 527® provides good adhesive attachment to the skin, ensures there are no air gaps, can be removed and reapplied, and provides good acoustic coupling even under dry skin conditions. Of course it is contemplated that other coupling materials with appropriate acoustic properties as well as other attachment could be used. As another example, the mixing properties of Sylgard 527® can be modified to balance rigidity/liquidity/tackiness. Generally, the coupling material needs to have low acoustic attenuation of ultrasonic wave and good acoustic contact, for example, a liquid like surface that can fill crevasses and contours of skin. The air pockets at contact surface must be avoided, for example, with a soft sticky material.

The transmitters/receivers 26, 28 are mounted in each module 24 at a predetermined orientation with respect to the patient's skin surface. In the alternative, a single transmitter and multiple receivers or multiple transmitters and a single receiver may be mounted within each module.

Generally, it is desirable to orient the transmitters/receivers 26, 28 in the module 24 so that, for example, the blood flow up the carotid artery into the brain is parallel to the orientation of the transmitters/receivers 26, 28.

In the exemplary embodiment, the sensor includes a plurality of modules. Even if the transmitter/receiver 26, 28 of one module is not adequately oriented to measure the blood flow, the remaining transmitters/receivers 26, 28 may adequately cover the carotid artery or other selected vessel and be positioned to measure the flow of the blood cells. Furthermore, multiple modules 24 reduce the risk of improperly securing the ultrasound assembly 10 on the patient. Even if the ultrasound assembly 10 is positioned slightly off, the multiplicity of transmitters/receivers 26, 28 ensures that at least one pair is sufficiently positioned over the carotid artery to provide the means to detect the pulse.

In the manner described above, the hands free collection of ultrasound sensor data from a subject is performed. The above is applicable to a variety of ultrasound imaging modalities such as B-mode, Color Doppler, CW Doppler, M-Mode, imaging applications, and non imaging applications such as pulse detection during a CPR guided intervention during a cardiac resuscitation attempt. As an example, the described above ultrasound assembly can measure the actual bloodflow during CPR to provide feedback regarding the quality of the CPR.

For example, the CPR unit 52 transmits the digital signals to the ultrasound sensor 12 which trigger the transmitters 26 to emit CW signals to the blood cells in the carotid artery. The reflected signals are received by the receivers 28. The return signals are indicative of the patient's pulse and thus, indicative of perfusion and are processed by the CPR unit to measure blood flow. For example, the return signals are compared with a threshold statistically appropriate with the returned signals received. If the return signals are above the threshold, then the return signals are indicative of a pulse and a rhythm is determined. Conversely, if the return signals are below the threshold, then the return signals are not considered a pulse but are considered to be background noise or low velocity residual flow. Based on the determined rhythm and absence of pulse, the CPR unit determines, for example, if performing CPR is advisable. The return signals may also be analyzed using appropriate signal processing methods including spectral analysis, correlation analysis and the like, in order to better infer the presence or absence of blood flow.

As another example, in nuclear medicine gating studies, such as cardiac imaging, the ultrasound assembly 10 is used to gate the data acquisition. The hands free ultrasound monitoring affords avoiding unnecessary exposure to radiation of the medical professional.

The apparatuses and methods described above are similarly applicable to animal imaging, monitoring, testing and treatment. Of course, it is contemplated that such apparatuses can be adopted for different size and/or anatomy of the animals, and the like.

With reference again to FIG. 1 and further reference to FIG. 4, the ultrasound assembly 10 is used in a health management system 110 which includes first and second stations 112, 114 and a host center 116. The first or care provider station 112 is located, for example, at a care provider site such as a physician's office or hospital and includes a terminal 120. One example of the terminal 120 is a personal computer which includes appropriate software 122, such as user interface software, and hardware 124, for interfacing with the host center 116 and via the host center 116 with the second station 114. The terminal 120 is connected to a first server 130 via an intranet or other connection as known in the art.

Of course, it is contemplated that the health management system 110 can include a plurality of the first stations 112, a plurality of host centers 116 and a plurality of second stations 114 as appropriate for an application.

A first link 140 provides the connection between the first station 112 and the host center 116. Alternatively, the first station 112 is a wireless station of a wireless local area network (LAN) or wireless wide are network (WAN).

The second or patient station 114 includes a user or patient interface 148 including a television set 150 or other patient display device which is located in a patient's home or dwelling. The user interface 148 further includes a control module, processor, algorithm or other means 152, such as set-top box, which interfaces with a video display 154 of the television set 150. The control module 152 converts and displays data from analog cable, digital cable, satellite, digital subscriber line (DSL), or digital broadcast television to a standard channel frequency, e.g. channel number, for display, for example, on a standard analog television set 150. In another embodiment, the module 152 converts and displays the data directly on the television set 150 via an RCA (Radio Corporation of America) or SCART (Syndicat des Constructeurs d'Appareils Radiorécepteurs et Téléviseurs) connector or interface, without the need to encode the signal onto a television channel frequency. In one embodiment, the control module 152 further receives on or off-air digital or conventional analog television signals from a cable or satellite provider or local broadcast TV for display on a DTV monitor. The control module 152 also receives signals such as digital or analog television format signals and patient information signals from the host center 116 via a second link 156. The examples of the second link 156 are wired connection, wireless connection, satellite connection, fiber optic connection, and the like.

The control module 152 is connected to the video display 154 via a switching device, algorithm or means 160 such as an audio/video (AV) switching device as known in the art. The switching device 160 provides switching between television reception from the tuner of the display 154 (or VCR, DVD or the like) and patient information reception/transmission from/to the host center 116. Alternatively, any other known type of input device adapted to provide an interface to the video display 154 is used.

For example, the patient information signals include information, instructions and queries that are displayed on the video display for information, action, and the like. The patient information signals include video and audio health issue programs, audio programs, video messages and audio messages, reminders to send health or biometric information, and the like. The user interface 148 further includes a remote interface device 166 which provides signals to an infrared transceiver 168. Signals from the transceiver 168 are provided to the control module 152 and function to select video input to the video display 154, input patient information, and the like. In one embodiment, the remote interface device 166 is a remote control device such as one commonly used in the home entertainment systems. In another embodiment, the remote interface device 166 is a computer input interface device, such as a keyboard or a mouse.

The host center 116 is centralized and includes various servers for specific functions. The examples of servers of the host center 116 are a video server 172, which provides pertinent video content to the display 154, and a measurement server 174. which collects and transfers patient's biometric measurements. The host center 116 includes a host center terminal 176 including appropriate hardware 178, software 180 and communications links 182 to enable connectivity between the first and second stations 112, 114.

It is also contemplated that the host center 116 is distributed, with different components or sub-centers hosting different functions. Alternatively, there may be a plurality of host centers 116 that connect a plurality of second stations 114 with one or more first stations 112.

In one embodiment, the second station 114 includes a set of medical devices 12 associated with the patient 14. E.g., in addition to the ultrasound assembly 10, the medical devices 12 can include a weight scale, a blood pressure device, an electrocardiogram, an electroencephalogram, an oximeter, a brain wave measuring device, a respiration monitor, a thermometer, and the like. In one embodiment, the biometric devices 12 are wireless devices which are worn by the patient 14 and communicate biometric reading continuously or at intervals to the host center, or are cabled devices which the patient 14 uses one or more times a day to take readings, or the like. Additionally, or alternatively, certain measurements may be manually entered by the patient via the remote device 166. Alternatively, the biometric device 12 can be implanted in the patient, such as a sensor on a pacemaker, on an infusion pump, and the like. Collected monitored or manual patient data are provided to a measurement gateway 184, which transmits the data to the measurement server 174 for processing and use.

Other exemplary user interface devices are a personal computer (PC), personal digital assistant (PDA), a mobile phone, a portable computer, automated voice response system and the like. As such, the display is accordingly a computer monitor, handheld communication device display, such as a portable phone, cellular phone or PDA.

The following is also applicable in the defibrillator, after shock detect presence/absence of PEA to determine if CPR should be performed, monitoring in ambulance from emergency location to hospital, noninvasive cardiovascular monitoring in hospital bed, hands-free bloodflow monitoring during surgery, possibly in presence of radiation from other imaging modalities or therapeutic treatments.

The invention has been described with reference to the preferred embodiments. Modifications and alterations may occur to others upon reading and understanding the preceding detailed description. It is intended that the invention be constructed as including all such modifications and alterations insofar as they come within the scope of the appended claims or the equivalents thereof.

1. A skin-mounted device comprising:
an acoustic module; and
an attachment assembly including an acoustically coupling layer, which affixes and acoustically couples the module to a skin.

2. The device as set forth in claim 1, wherein the acoustic module includes an ultrasonic transducer.

3. The device as set forth in claim 1, further including a plurality of modules affixed and acoustically coupled to the skin by the coupling layer.

4. The device as set forth in claim 3, further including:
a flexible covering layer which surroundingly extends over the modules to permit flexing for conforming to the contour of the skin.

5. The device as set forth in claim 1, wherein the attachment assembly further includes:
an adhesive pad which extends peripherally around the coupling layer.

6. The device as set forth in claim 5 further including:
a covering layer which covers an opposite side of the module from the coupling layer, an inner portion of the adhesive pad being sandwiched between the covering and coupling layers.

7. The device as set forth in claim 1, wherein the coupling layer includes:
a dielectric silicone gel which is sufficiently tacky to adhere to the skin and sufficiently fluid to conform to the skin and fissures therein without air pockets.

8. The device as set forth in claim 1, wherein the acoustic module includes at least one of B-mode, Color Doppler, CW Doppler, and M-Mode ultrasound sensor.

9. The device as set forth in claim 1, wherein the acoustic module includes a wireless transducer.

10. The device as set forth in claim 1, wherein the acoustic module includes:
an acoustic transmitter element, an acoustic receiver element, and a former which holds the acoustic transmitter and receiver elements in a fixed relationship to each other.

11. The device as set forth in claim 10, further including:
a plurality of the modules flexibly interconnected between the acoustic coupling layer on one side and a flexible covering layer on the other side.

12. The device as set forth in claim 11, wherein at least one of the covering and coupling layers includes Sylgard® dielectric gel.

13. The device as set forth in claim 11, wherein the coupling layer includes Sylgard® dielectric gel and the covering layer includes T2® silicone.

14. The device as set forth in claim 11, wherein the attachment assembly further includes:
a pad with which the covering and coupling layers are integrated.

15. The device as set forth in claim 11, wherein the coupling layer has a sufficiently low sound speed and wherein the skin-mounted device is substantially flat.

16. The device as set forth in claim 1, wherein the module includes an array of piezoelectric modules each including:
a transmitter which generates an ultrasonic transmission directed at a moving substance below the skin, and
a receiver which senses reflected ultrasonic transmission which is indicative of the motion.

17. The device as set forth in claim 1, wherein the module includes an array of piezoelectric modules each including:
a transceiver which generates an ultrasonic transmission directed at a moving substance below the skin and senses reflected ultrasonic transmission which is indicative of the motion.

18. The device as set forth in claim 1 in combination with a cardiac defibrillation unit.

19. The device as set forth in claim 1, wherein the coupling layer is (a) sufficiently tacky to adhere the module to the skin and hold the module in place without further human intervention during a medical procedure, (b) sufficiently firm to prevent slippage, and (c) sufficiently fluid to fill fissures in the skin as it is affixed.

20. A nuclear imager in which the device of claim 1 is used to gate the data acquisition.

21. A method of acoustic monitoring comprising:
affixing and acoustically coupling an acoustic module with a patient's skin with an acoustic coupling layer;
transmitting and receiving acoustic signals with the module to monitor a physiological parameter of the patient.

22. The method as set forth in claim 21, wherein the physiological parameter includes blood flow.

23. The method as set forth in claim 21, wherein an array of acoustic modules are sandwiched between the coupling layer and a flexible covering layer and wherein the affixing step includes flexing the covering and coupling layers to conform the array to a patient contour.

24. The method as set forth in claim 21, wherein the coupling layer is (a) sufficiently tacky to adhere the module to the skin and hold the module in place without further human intervention during a medical procedure, (b) sufficiently firm to prevent slippage, and (c) sufficiently fluid to fill fissures in the skin as it is affixed.

25. The method as set forth in claim 24, wherein the coupling layer includes an adhesive silicone gel.

26. An apparatus comprising:
a coupling means for affixing and acoustically coupling an acoustic module with a patient's skin; and
a medical means for transmitting and receiving acoustic signals to monitor a physiological parameter of the patient.

27. A monitoring system comprising:
an acoustic module which senses a physiological parameter of a patient;
an attachment assembly including an acoustically coupling layer, which affixes and acoustically couples the module to a skin of the patient; and
a server which collects the sensed patient data and transfers the sensed patient data to a monitoring station.
